(19) United States
(12) Patent Application Publication
(10) Pub. No.: US 2026/0000373 A1
(43) Pub. Date: Jan. 1, 2026

(54) MEDICAL DIAGNOSTIC DEVICE, DATA CONTROL METHOD, STORAGE MEDIUM STORING

(71) Applicant: FUJIFILM Corporation, Tokyo (JP)

(72) Inventor: Taiga GOTO, Tokyo (JP)

(73) Assignee: FUJIFILM Corporation, Tokyo (JP)

(21) Appl. No.: 19/239,996

(22) Filed: Jun. 17, 2025

(30) Foreign Application Priority Data
Jun. 27, 2024 (JP) 2024-104029

(51) Int. Cl. A61B 6/42; A61B 6/00

(52) U.S. Cl. CPC A61B 6/4241; A61B 6/5258

(57) ABSTRACT

Provided are a medical diagnostic device, a data control method, and a storage medium storing that are less likely to be affected by pile-up even during imaging with a high dose.

A medical diagnostic device includes at least one processor, in which the processor acquires positioning imaging data obtained by scanning a subject with a first dose before main imaging of the subject and main imaging data obtained by performing the main imaging on the subject with a second dose higher than the first dose, and corrects the main imaging data by specific processing using at least the positioning imaging data, to output corrected imaging data.

### CROSS-REFERENCE TO RELATED APPLICATION

This application claims priority from Japanese Patent Application No. 2024-104029 filed on Jun. 27, 2024, the entire disclosure of which is incorporated herein by reference.

### BACKGROUND

### 1. Technical Field

The technology of the present disclosure relates to a medical diagnostic device, a data control method, and a storage medium storing.

### 2. Description of the Related Art

Development of a photon counting computed tomography (PCCT) apparatus comprising a detector adopting a photon counting method is in progress (for example, JP2024-053288A). The detector identifies energy (wavelength) of incident radiation and counts the number of times the radiation is detected for each of a plurality of energy levels. The PCCT apparatus can obtain more information than a computed tomography (CT) apparatus equipped with a charge integration type detector.

### SUMMARY

However, since the detector detects a plurality of radiation photons as a single radiation photon, the detector is likely to be affected by the pile-up during imaging (main scanning) in which a high dose is used to collect an image used for diagnosis. That is, during the main scanning, an error may occur in imaging data due to an erroneous determination of the number of times of the radiation is detected and the energy of the incident radiation.

In view of such a situation, an object of the present disclosure is to provide a medical diagnostic device, a data control method, and a storage medium storing that are less likely to be affected by pile-up even during imaging with a high dose.

In order to achieve the above-described object, the technology of the present disclosure provides a medical diagnostic device comprising: at least one processor, in which the processor acquires positioning imaging data obtained by scanning a subject with a first dose before main imaging of the subject and main imaging data obtained by performing the main imaging on the subject with a second dose higher than the first dose, and corrects the main imaging data by specific processing using at least the positioning imaging data, to output corrected imaging data.

The processor acquires, as the main imaging data, data obtained by synchronizing an imaging trajectory during the main imaging with an imaging trajectory during the scanning.

In the specific processing, the processor performs forward projection of a positioning image included in the acquired positioning imaging data in accordance with a trace of the main imaging data, to generate forward projection data of the positioning imaging data, and corrects the main imaging data using the generated forward projection data.

In the specific processing, the processor corrects the main imaging data by using a difference in imaging data between the positioning imaging data and the main imaging data.

In the specific processing, the processor corrects the main imaging data by using a difference between imaging data portions other than high-frequency noise included in each of the positioning imaging data and the main imaging data.

In the specific processing, the processor corrects the main imaging data by using a difference between imaging data portions in which high-frequency noise included in each of the positioning imaging data and the main imaging data is removed.

In the specific processing, the processor corrects the main imaging data by using a difference between an imaging data portion in which noise included in the positioning imaging data is removed and the main imaging data.

In the specific processing, the processor estimates a magnitude of an error during the main imaging based on the number of photons or a projection value detected by a detector, and corrects the main imaging data using the positioning imaging data such that the estimated error is removed.

In the specific processing, in a case in which a difference in imaging data between the positioning imaging data and the main imaging data tends to be large, the processor corrects the main imaging data by multiplying the main imaging data by a coefficient corresponding to a ratio between the positioning imaging data and the main imaging data.

In the specific processing, the processor corrects the main imaging data by multiplying the main imaging data by a coefficient corresponding to a ratio between the positioning imaging data with a tendency for a low projection value during the scanning and the main imaging data.

In the specific processing, the processor corrects the main imaging data such that a specific representative value of the positioning imaging data and the main imaging data becomes the same in both the positioning imaging data and the main imaging data.

In the specific processing, the processor corrects the main imaging data by mixing a low-frequency component included in the positioning imaging data and a high-frequency component included in the main imaging data.

In the specific processing, the processor compares low-frequency components included in each of the positioning imaging data and the main imaging data, and corrects the main imaging data based on a comparison result such that the low-frequency component included in the main imaging data becomes close to the low-frequency component included in the positioning imaging data.

In the specific processing, the processor reconstructs a tomographic image of the subject by blending the positioning imaging data and the corrected main imaging data.

The technology of the present disclosure provides a data control method executed by a computer, the data control method comprising: acquiring positioning imaging data obtained by scanning a subject with a first dose before main imaging of the subject and main imaging data obtained by performing the main imaging on the subject with a second dose higher than the first dose; and correcting the main imaging data by specific processing using at least the positioning imaging data, to output corrected imaging data.

The technology of the present disclosure provides a non-transitory computer-readable storage medium storing a data control program causing a computer to execute a process comprising: acquiring positioning imaging data obtained by scanning a subject with a first dose before main imaging of the subject and main imaging data obtained by performing the main imaging on the subject with a second dose higher than the first dose; and correcting the main imaging data by specific processing using at least the positioning imaging data, to output corrected imaging data.

According to the technology of the present disclosure, it is possible to reduce the influence of the pile-up even during imaging with a high dose.

### BRIEF DESCRIPTION OF THE DRAWINGS

FIG. 1 is a configuration diagram of a medical diagnostic system 100 according to an embodiment of the present disclosure.

FIG. 2 is a diagram showing an example of a hardware configuration of a medical diagnostic device 4 according to the embodiment of the present disclosure.

FIG. 3 is a diagram showing a functional block of the medical diagnostic device 4 according to the embodiment of the present disclosure.

FIG. 4 is a flowchart showing an operation of the medical diagnostic device 4.

### DETAILED DESCRIPTION

Hereinafter, an example of an embodiment of the present disclosure will be described with reference to the accompanying drawings. It should be noted that, in each drawing, the same or equivalent constituent elements and parts are denoted by the same reference numerals. In addition, dimensional ratios in the drawings are exaggerated for convenience of description, and may be different from the actual ratios.

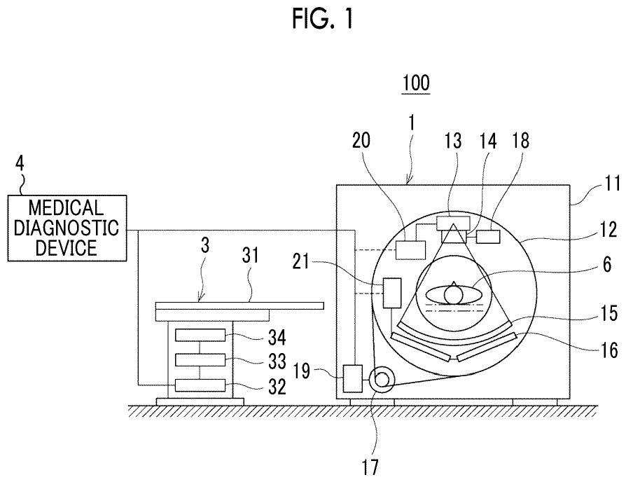

FIG. 1 is a configuration diagram of a medical diagnostic system 100 according to the embodiment of the present disclosure. The medical diagnostic system 100 may be interpreted as, for example, an X-ray CT apparatus. It should be noted that the medical diagnostic system 100 is not limited to the X-ray CT apparatus, and may be a system related to other medical examinations. The medical diagnostic system 100 may comprise a scanner 1, an patient table 3, and a medical diagnostic device 4.

### Scanner 1

The scanner 1 may be interpreted as a unit that executes CT scanning. The scanner 1 may comprise a stand 11 as a gantry, a rotation plate 12, an X-ray tube device 13, a collimator 14, an X-ray detector 15, and a rotation plate drive device 17.

The rotation plate 12 may be interpreted as a plate that has an opening at a central portion and is rotatably supported by the stand 11. The rotation plate 12 may comprise a collimator control device 18, a rotation plate drive control device 19, an X-ray high-voltage generation device 20, a data collection device 16, and a data transmission device 21.

The collimator control device 18 may control the collimator 14 to change an X-ray irradiation field. The rotation plate drive control device 19 may perform drive control of the rotation plate drive device 17. The X-ray high-voltage generation device 20 may supply power for X-ray generation to the X-ray tube device 13 and control an X-ray generation condition. The data collection device 16 may collect the output of the X-ray detector 15. The data transmission device 21 may transmit the data collected by the data collection device 16. It should be noted that the supply of the power and the control signal to each unit provided in the rotation plate 12 and taking-out of the data from each unit provided in the rotation plate 12 are performed via a slip ring (not shown) provided between the stand 11 and the rotation plate 12.

The X-ray tube device 13 may be fixed to the rotation plate 12. The collimator 14 may be provided in an X-ray radiation port portion of the X-ray tube device 13. The X-ray detector 15 may be disposed to face the X-ray tube device 13 with the opening of the rotation plate 12 interposed therebetween. The rotation plate drive device 17 may be provided in the stand 11. The X-ray detector 15 may be interpreted as a detector adopting a photon counting method.

### Examination Table 3

The patient table 3 may be interpreted as a table that moves a subject 6 between an imaging preparation position and an imaging position. The subject 6 is disposed on a top plate 31. Although not shown, an up-down movement mechanism and a front-rear movement mechanism of the top plate 31 are provided. In addition, the patient table 3 is provided with an patient table control device 32, a top plate up-down movement control device 33, and a top plate front-rear movement control device 34 for controlling operations of the up-down movement mechanism and the front-rear movement mechanism of the top plate 31.

### Medical Diagnostic Device 4

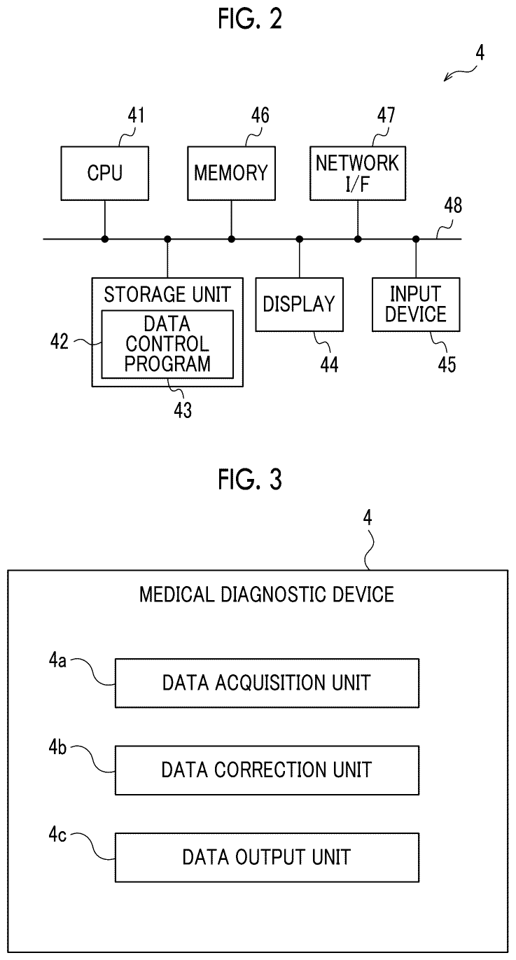

Next, an example of a hardware configuration of the medical diagnostic device 4 will be described with reference to FIG. 2. FIG. 2 is a diagram showing an example of the hardware configuration of the medical diagnostic device 4 according to the embodiment of the present disclosure. The medical diagnostic device 4 may comprise a central processing unit (CPU) 41, a storage unit 43 that is a non-volatile memory, a display 44 such as a liquid crystal display, an input device 45, a memory 46 that is a transitory storage region, and a network interface (I/F) 47. The CPU 41, the memory 46, the storage unit 43, the display 44, the input device 45, and the network I/F 47 are connected to a bus 48.

The CPU 41 may be interpreted as a processor according to the present disclosure. The input device 45 may include a pointing device, such as a keyboard and a mouse. The storage unit 43 can be implemented by a hard disk drive (HDD), a solid state drive (SSD), a flash memory, or the like. The storage unit 43 may store a data control program 42. The CPU 41 may read out the data control program 42 from the storage unit 43, load the data control program 42 into the memory 46, and execute the loaded data control program 42. The data control program 42 may be provided in a cloud server and the like. The storage unit 43 is a non-transitory computer-readable storage medium storing a data control program for causing a computer to execute a process.

Next, functions of the medical diagnostic device 4 will be described with reference to FIG. 3. FIG. 3 is a diagram showing a functional block of the medical diagnostic device 4 according to the embodiment of the present disclosure. The medical diagnostic device 4 may comprise a data acquisition unit 4a, a data correction unit 4b, and a data output unit 4c. The functional configuration may be implemented by reading out the data control program 42 and loading the data control program 42 into the memory 46 via the CPU 41 shown in FIG. 2.

### Data Acquisition Unit 4a

The data acquisition unit 4a may acquire positioning imaging data obtained by scanning the subject 6 with a first dose before the main imaging of the subject 6, and main imaging data obtained by performing main imaging of the subject 6 with a second dose higher than the first dose.

The positioning imaging data may include projection data obtained by imaging for collecting a positioning image (scan image), X-ray measurement data (detector measurement value), and the like. The main imaging data may include projection data obtained by main imaging (scanning) for collecting an image used for observation, X-ray measurement data (detector measurement value), and the like.

### Data Acquisition Example

The data acquisition unit 4a may acquire, as the main imaging data, data obtained by synchronizing an imaging trajectory during the main imaging with an imaging trajectory (trace) during the scanning.

Specifically, the main imaging data may be acquired in a case in which a trajectory (trace) of an X-ray source in helical imaging during the main imaging is synchronized with a trajectory (trace) of an X-ray source in helical imaging during the scanning. It should be noted that the subject for which the positioning imaging data is captured with a low dose and the subject for which the main imaging data is captured with a high dose are acquired may be the same subject.

A detection ratio of X-rays is significantly different between the positioning imaging data captured with a low dose and the main imaging data captured with a high dose, but the projection data (a value obtained by multiplying an attenuation coefficient of X-rays by a path length of each transmitted substance) is originally the same. The data correction unit 4b may correct the main imaging data using, for example, a difference between the main imaging data and the positioning imaging data synchronized with the imaging (trace). The configuration of the data correction unit 4b will be described in detail later.

### Data Correction Unit 4b

The data correction unit 4b may correct the main imaging data by specific processing using at least the positioning imaging data. Hereinafter, a specific example of the data correction via the data correction unit 4b will be described.

### Data Correction Example 1

In the specific processing, the data correction unit 4b may generate forward projection data of the acquired positioning imaging data by performing forward projection of the positioning image included in the acquired positioning imaging data in accordance with the trace of the main imaging data, to correct the main imaging data using the generated forward projection data.

Specifically, the data correction unit 4b obtains the forward projection data in which the trace matches the trace of the main imaging data (main imaging data) by performing re-imaging (forward projection) processing on low-dose imaging data (positioning imaging data). The forward projection data obtained from the low-dose imaging data is imaging data in which an error is less likely to occur in the imaging data. The data correction unit 4b may obtain, for example, a difference, a ratio, or the like between the forward projection data and the main imaging data, which have the matching trace, based on the forward projection data, to correct the main imaging data such that the difference, the ratio, or the like becomes small.

### Data Correction Example 2

In the specific processing, the data correction unit 4b may correct the main imaging data by using a difference in the imaging data between the positioning imaging data and the main imaging data.

For example, in the specific processing, the data correction unit 4b may correct the main imaging data by using a difference between imaging data portions other than high-frequency noise included in each of the positioning imaging data (or the forward projection data) and the main imaging data. By obtaining the difference in the imaging data between the positioning imaging data and the main imaging data, an error of the remaining components excluding the high-frequency noise included in the main imaging data can be extracted. The data correction unit 4b may regard the positioning imaging data captured with a low dose as correct data and correct the main imaging data such that an error of remaining components excluding the high-frequency noise included in the main imaging data becomes small.

### Data Correction Example 4

In the specific processing, the data correction unit 4b may correct the main imaging data by using a difference between imaging data portions (for example, low and medium frequency regions of the imaging data) in which high-frequency noise included in each of the positioning imaging data and the main imaging data is removed.

In the specific processing, the data correction unit 4b may correct the main imaging data by using a difference between imaging data portions in which high-frequency noise included in each of the forward projection data and the main imaging data is removed, instead of the positioning imaging data and the main imaging data.

### Data Correction Example 5

In the specific processing, the data correction unit 4b may correct the main imaging data by using a difference between the main imaging data and an imaging data portion in which noise included in the positioning imaging data is removed.

Specifically, the data correction unit 4b may reduce noise included in the positioning imaging data by channel averaging processing, and correct the main imaging data by using a difference between the positioning imaging data after the noise is reduced and the imaging data portion (for example, the low and medium frequency ranges of the imaging data).

In the specific processing, the data correction unit 4b may reduce the noise included in the forward projection data by the channel averaging processing instead of the noise included in the positioning imaging data, and correct the main imaging data using a difference between the forward projection data after the noise is reduced and the imaging data portion (for example, the low and medium frequency ranges of the imaging data).

### Data Correction Example 6

In the specific processing, the data correction unit 4b may estimate a magnitude of the error during the main imaging based on the number of photons or a projection value detected by the detector (X-ray detector 15), and correct the main imaging data using the positioning imaging data such that the estimated error is removed. The number of photons may be interpreted as the number of photons detected by the X-ray detector 15. The projection value may be interpreted as a value output by a detection element included in the X-ray detector 15 in accordance with the number of photons.

In the PCCT, the number of X-ray photons can be detected, and a ratio of the number of X-ray photons included in each of the positioning imaging data and the main imaging data is a value corresponding to a ratio of the X-ray dose. For example, in a case in which a ratio between a dose of low-dose imaging data and a dose of high-dose imaging data is 1:2, a ratio of the number of X-ray photons is also 1:2. Since there is a correlation between the error of the high-dose imaging data and the error of the low-dose imaging data, the data correction unit 4b can estimate the magnitude of the error in the main imaging in accordance with the ratio of the number of X-ray photons. The data correction unit 4b may correct the main imaging data using the positioning imaging data such that the estimated error is removed.

### Data Correction Example 7

In the specific processing, in a case in which there is a large difference in the imaging data between the positioning imaging data and the main imaging data, the data correction unit 4b may correct the main imaging data by multiplying the main imaging data by a coefficient corresponding to the ratio between the positioning imaging data and the main imaging data. The case in which the difference in the imaging data tends to be large may be interpreted as a case in which the X-ray dose detected by the X-ray detector 15 is at a level affected by the pile-up, that is, a level at which an error may occur in the imaging data. The coefficient corresponding to the ratio between the positioning imaging data and the main imaging data may be interpreted as a ratio between the number of X-ray photons in the above-described example.

### Data Correction Example 8

In the specific processing, the data correction unit 4b may correct the main imaging data by multiplying the main imaging data by a coefficient corresponding to a ratio between the main imaging data with a tendency for a low projection value during the scanning and the positioning imaging data. During the scanning (low-dose imaging), the X-ray dose transmitted through these parts changes depending on the type of the part (heart, bone, or the like) of the subject 6. In a case in which the number of X-ray photons detected by the detector (X-ray detector 15) is large, the projection value is low, and thus the projection value is likely to be affected by the pile-up. Therefore, the data correction unit 4b may use the imaging data that is likely to be affected by the pile-up, that is, the positioning imaging data in which the projection value is low during the low-dose imaging, and multiply the main imaging data by a coefficient corresponding to the ratio between the data and the main imaging data.

### Data Correction Example 9

In the specific processing, the data correction unit 4b may correct the main imaging data such that a specific representative value of the positioning imaging data and the main imaging data becomes the same in both the positioning imaging data and the main imaging data. The representative value may be interpreted as, for example, an average value of the imaging data in a specific-frequency component (for example, a low-frequency component) included in the imaging data and components (medium and high-frequency components) around the specific-frequency component.

### Data Correction Example 10

In the specific processing, the data correction unit 4b may correct the main imaging data by mixing the low-frequency component included in the positioning imaging data and the high-frequency component included in the main imaging data. The mixing of the low-frequency component included in the positioning imaging data and the high-frequency component included in the main imaging data may be interpreted as a combination of the low-frequency component of the low-dose imaging data that is less likely to be affected by the pile-up and the high-frequency component of the high-dose imaging data that is less likely to be affected by the noise corresponding to the X-ray dose. The positioning imaging data includes a high-frequency error, and the main imaging data includes a low-frequency error. Therefore, the data correction unit 4b improves the correction accuracy of the main imaging data by using the low-frequency component of the positioning imaging data and the high-frequency component of the main imaging data.

### Data Correction Example 11

In the specific processing, the data correction unit 4b may compare the low-frequency components included in each of the positioning imaging data and the main imaging data with each other, and correct the main imaging data based on the comparison result such that the low-frequency component included in the main imaging data becomes close to the low-frequency components included in the positioning imaging data. Specifically, the data correction unit 4b may compare, for example, the low-frequency component in the low-dose imaging and the low-frequency component in the high-dose imaging using the fact that the correction amounts are the same in data having similar projection values in the positioning imaging data and the main imaging data, and correct the main imaging data such that the low-frequency component in the high-dose imaging becomes close to the low-frequency component in the low-dose imaging.

### Data Correction Example 12

In the specific processing, the data correction unit 4b may reconstruct the tomographic image of the subject 6 by blending the positioning imaging data and the corrected main imaging data. Specifically, low-frequency information of the positioning imaging data and high-frequency information of the main imaging data are mixed. More specifically, since the noise is the high-frequency information, the main imaging data captured with a high dose is used, and since the error such as the pile-up is relatively low-frequency information, the blending is performed using the low-frequency information of the positioning imaging data in which the error such as the pile-up is small. In this way, by reconstructing the tomographic image is reconstructed, a tomographic image with a good image quality can be acquired. It should be noted that the data to be corrected may include normal image data, spectral data (base substance data, virtual monochromatic data, and energy discrimination data (substance discrimination image data)), and the like.

### Data Output Unit 4c

The data output unit 4c may output the data corrected by the data correction unit 4b, that is, the corrected imaging data.

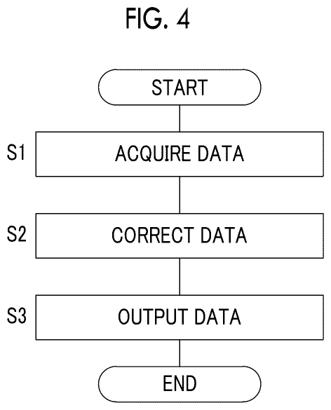

Next, the operation of the medical diagnostic device 4 will be described with reference to FIG. 4. FIG. 4 is a flowchart showing the operation of the medical diagnostic device 4.

In step S1, the data acquisition unit 4a may acquire the positioning imaging data obtained by scanning the subject 6 with the first dose before the main imaging of the subject 6, and the main imaging data obtained by performing the main imaging of the subject 6 with the second dose higher than the first dose. In step S2, the data correction unit 4b may correct the main imaging data by the specific processing using at least the positioning imaging data. In step S3, the data output unit 4c may output the data corrected by the data correction unit 4b, that is, the corrected imaging data.

As described above, the medical diagnostic device 4 according to the present disclosure acquires positioning imaging data obtained by scanning the subject 6 with a first dose before main imaging of the subject 6 and main imaging data obtained by performing the main imaging on the subject 6 with a second dose higher than the first dose, and corrects the main imaging data by specific processing using at least the positioning imaging data, to output corrected imaging data.

With the medical diagnostic device 4 according to the present disclosure, even in a case in which the number of times of the radiation is detected and the energy of the incident radiation are erroneously determined during the main scanning, the high-dose imaging data can be corrected by using the low-dose imaging data that is less likely to be affected by the pile-up, so that the error included in the imaging data can be reduced.

In the above-described embodiment, for example, the following various processors can be used as a hardware structure of the processing units that execute various types of processing, such as the data acquisition unit 4a, the data correction unit 4b, and the data output unit 4c. As described above, the various processors include, in addition to the CPU that is a general-purpose processor that executes software (program) to function as various processing units, a programmable logic device (PLD) that is a processor of which a circuit configuration can be changed after manufacture, such as a field-programmable gate array (FPGA), a graphics processing unit (GPU), a dedicated electric circuit that is a processor having a circuit configuration dedicatedly designed to execute specific processing, such as an application specific integrated circuit (ASIC), and the like.

One processing unit may be configured by one of the various processors or may be configured by combining two or more processors of the same type or different types (for example, by combining a plurality of FPGAs or combining a CPU and an FPGA). A plurality of processing units may be configured by one processor.

A first example of the configuration in which the plurality of processing units are configured by one processor is a form in which one processor is configured by a combination of one or more CPUs and the software and this processor functions as the plurality of processing units, as represented by computers such as a client and a server. Second, as represented by a system-on-a-chip (SoC) or the like, there is a form in which a processor, which implements the functions of the entire system including the plurality of processing units with a single integrated circuit (IC) chip, is used. As described above, as the hardware structure, the various processing units are configured by one or more of the various processors described above.

Furthermore, as the hardware structure of the various processors, more specifically, an electric circuit (circuitry) in which circuit elements, such as semiconductor elements, are combined can be used.

In addition, in the above-described embodiment, the aspect has been described in which the data control program 42 is stored (installed) in the memory 46 in advance, but the present disclosure is not limited to this. The data control program 42 may be provided in a form of being recorded in a recording medium such as a compact disc read only memory (CD-ROM), a digital versatile disc read only memory (DVD-ROM), and a universal serial bus (USB) memory. In addition, the data control program 42 may be provided in a form of being downloaded from an external device through a network.

The technology of the present disclosure extends to a computer-readable storage medium (CD-ROM, DVD-ROM, USB memory, or the like) that non-transitorily stores the data control program 42, in addition to the data control program 42.

In addition, the present disclosure is not limited to the above-described embodiment, and various modifications can be made without departing from the gist of the present disclosure. The present invention can also be applied to a program product.

In regard to the above-described embodiment, the following supplementary notes will be further disclosed.

### Supplementary Note 1

A medical diagnostic device comprising: at least one processor, in which the processor acquires positioning imaging data obtained by scanning a subject with a first dose before main imaging of the subject and main imaging data obtained by performing the main imaging on the subject with a second dose higher than the first dose, and corrects the main imaging data by specific processing using at least the positioning imaging data, to output corrected imaging data.

### Supplementary Note 2

The medical diagnostic device according to supplementary note 1, in which the processor acquires, as the main imaging data, data obtained by synchronizing an imaging trajectory during the main imaging with an imaging trajectory during the scanning.

### Supplementary Note 3

The medical diagnostic device according to supplementary note 1 or 2, in which, in the specific processing, the processor performs forward projection of a positioning image included in the acquired positioning imaging data in accordance with a trace of the main imaging data, to generate forward projection data of the positioning imaging data, and corrects the main imaging data using the generated forward projection data.

### Supplementary Note 4

The medical diagnostic device according to any one of supplementary notes 1 to 3, in which, in the specific processing, the processor corrects the main imaging data by using a difference in imaging data between the positioning imaging data and the main imaging data.

### Supplementary Note 5

The medical diagnostic device according to any one of supplementary notes 1 to 4, in which, in the specific processing, the processor corrects the main imaging data by using a difference between imaging data portions other than high-frequency noise included in each of the positioning imaging data and the main imaging data.

### Supplementary Note 6

The medical diagnostic device according to any one of supplementary notes 1 to 5, in which, in the specific processing, the processor corrects the main imaging data by using a difference between imaging data portions in which high-frequency noise included in each of the positioning imaging data and the main imaging data is removed.

### Supplementary Note 7

The medical diagnostic device according to any one of supplementary notes 1 to 6, in which, in the specific processing, the processor corrects the main imaging data by using a difference between an imaging data portion in which noise included in the positioning imaging data is removed and the main imaging data.

### Supplementary Note 8

The medical diagnostic device according to any one of supplementary notes 1 to 7, in which, in the specific processing, the processor estimates a magnitude of an error during the main imaging based on the number of photons or a projection value detected by a detector, and corrects the main imaging data using the positioning imaging data such that the estimated error is removed.

### Supplementary Note 9

The medical diagnostic device according to any one of supplementary notes 1 to 8, in which, in the specific processing, in a case in which a difference in imaging data between the positioning imaging data and the main imaging data tends to be large, the processor corrects the main imaging data by multiplying the main imaging data by a coefficient corresponding to a ratio between the positioning imaging data and the main imaging data.

### Supplementary Note 10

The medical diagnostic device according to any one of supplementary notes 1 to 9, in which, in the specific processing, the processor corrects the main imaging data by multiplying the main imaging data by a coefficient corresponding to a ratio between the positioning imaging data with a tendency for a low projection value during the scanning and the main imaging data.

### Supplementary Note 11

The medical diagnostic device according to supplementary note 10, in which, in the specific processing, the processor corrects the main imaging data such that a specific representative value of the positioning imaging data and the main imaging data becomes the same in both the positioning imaging data and the main imaging data.

### Supplementary Note 12

The medical diagnostic device according to any one of supplementary notes 1 to 11, in which, in the specific processing, the processor corrects the main imaging data by mixing a low-frequency component included in the positioning imaging data and a high-frequency component included in the main imaging data.

### Supplementary Note 13

The medical diagnostic device according to any one of supplementary notes 1 to 12, in which, in the specific processing, the processor compares low-frequency components included in each of the positioning imaging data and the main imaging data, and corrects the main imaging data based on a comparison result such that the low-frequency component included in the main imaging data becomes close to the low-frequency component included in the positioning imaging data.

### Supplementary Note 14

The medical diagnostic device according to any one of supplementary notes 1 to 13, in which, in the specific processing, the processor reconstructs a tomographic image of the subject by blending the positioning imaging data and the corrected main imaging data.

### Supplementary Note 15

A data control method executed by a computer, the data control method comprising: acquiring positioning imaging data obtained by scanning a subject with a first dose before main imaging of the subject and main imaging data obtained by performing the main imaging on the subject with a second dose higher than the first dose; and correcting the main imaging data by specific processing using at least the positioning imaging data, to output corrected imaging data.

### Supplementary Note 16

A non-transitory computer-readable storage medium storing a data control program causing a computer to execute: acquire positioning imaging data obtained by scanning a subject with a first dose before main imaging of the subject and main imaging data obtained by performing the main imaging on the subject with a second dose higher than the first dose; and correct the main imaging data by specific processing using at least the positioning imaging data, to output corrected imaging data.

### Supplementary Note 17

A computer program product including a program causing at least one processor to execute a process comprising: acquiring positioning imaging data obtained by scanning a subject with a first dose before main imaging of the subject and main imaging data obtained by performing the main imaging on the subject with a second dose higher than the first dose; and correcting the main imaging data by specific processing using at least the positioning imaging data, to output corrected imaging data.

What is claimed is:

1. A medical diagnostic device comprising:
at least one processor,
wherein the processor
acquires positioning imaging data obtained by scanning a subject with a first dose before main imaging of the subject and main imaging data obtained by performing the main imaging on the subject with a second dose higher than the first dose, and
corrects the main imaging data by specific processing using at least the positioning imaging data, to output corrected imaging data.

2. The medical diagnostic device according to claim 1,
wherein the processor acquires, as the main imaging data, data obtained by synchronizing an imaging trajectory during the main imaging with an imaging trajectory during the scanning.

3. The medical diagnostic device according to claim 1,
wherein, in the specific processing, the processor performs forward projection of a positioning image included in the acquired positioning imaging data in accordance with a trace of the main imaging data, to generate forward projection data of the positioning imaging data, and corrects the main imaging data using the generated forward projection data.

4. The medical diagnostic device according to claim 1,
wherein, in the specific processing, the processor corrects the main imaging data by using a difference in imaging data between the positioning imaging data and the main imaging data.

5. The medical diagnostic device according to claim 1,
wherein, in the specific processing, the processor corrects the main imaging data by using a difference between imaging data portions other than high-frequency noise included in each of the positioning imaging data and the main imaging data.

6. The medical diagnostic device according to claim 1,
wherein, in the specific processing, the processor corrects the main imaging data by using a difference between imaging data portions in which high-frequency noise included in each of the positioning imaging data and the main imaging data is removed.

7. The medical diagnostic device according to claim 1,
wherein, in the specific processing, the processor corrects the main imaging data by using a difference between an imaging data portion in which noise included in the positioning imaging data is removed and the main imaging data.

8. The medical diagnostic device according to claim 1,
wherein, in the specific processing, the processor estimates a magnitude of an error during the main imaging based on the number of photons or a projection value detected by a detector, and corrects the main imaging data using the positioning imaging data such that the estimated error is removed.

9. The medical diagnostic device according to claim 1,
wherein, in the specific processing, in a case in which a difference in imaging data between the positioning imaging data and the main imaging data tends to be large, the processor corrects the main imaging data by multiplying the main imaging data by a coefficient corresponding to a ratio between the positioning imaging data and the main imaging data.

10. The medical diagnostic device according to claim 1,
wherein, in the specific processing, the processor corrects the main imaging data by multiplying the main imaging data by a coefficient corresponding to a ratio between the positioning imaging data with a tendency for a low projection value during the scanning and the main imaging data.

11. The medical diagnostic device according to claim 10,
wherein, in the specific processing, the processor corrects the main imaging data such that a specific representative value of the positioning imaging data and the main imaging data becomes the same in both the positioning imaging data and the main imaging data.

12. The medical diagnostic device according to claim 1,
wherein, in the specific processing, the processor corrects the main imaging data by mixing a low-frequency component included in the positioning imaging data and a high-frequency component included in the main imaging data.

13. The medical diagnostic device according to claim 1,
wherein, in the specific processing, the processor compares low-frequency components included in each of the positioning imaging data and the main imaging data, and corrects the main imaging data based on a comparison result such that the low-frequency component included in the main imaging data becomes close to the low-frequency component included in the positioning imaging data.

14. The medical diagnostic device according to claim 1,
wherein, in the specific processing, the processor reconstructs a tomographic image of the subject by blending the positioning imaging data and the corrected main imaging data.

15. A data control method executed by a computer, the data control method comprising:
acquiring positioning imaging data obtained by scanning a subject with a first dose before main imaging of the subject and main imaging data obtained by performing the main imaging on the subject with a second dose higher than the first dose; and
correcting the main imaging data by specific processing using at least the positioning imaging data, to output corrected imaging data.

16. A non-transitory computer-readable storage medium storing a data control program causing a computer to execute:
acquire positioning imaging data obtained by scanning a subject with a first dose before main imaging of the subject and main imaging data obtained by performing the main imaging on the subject with a second dose higher than the first dose; and
correct the main imaging data by specific processing using at least the positioning imaging data, to output corrected imaging data.
